(19) United States
(12) Patent Application Publication
(10) Pub. No.: US 2026/0232356 A1
(43) Pub. Date: Aug. 13, 2026

(54) MULTIAXIAL RECEIVERS WITH TETHER

(71) Applicant: Warsaw Orthopedic, Inc., Warsaw, IN (US)

(72) Inventors: Jerald L. Redmond, Germantown, TN (US); William Alan Rezach, Covington, TN (US)

(73) Assignee: Warsaw Orthopedic, Inc., Warsaw, IN (US)

(21) Appl. No.: 19/159,236

(22) PCT Filed: Feb. 21, 2024
(86) PCT No.: PCT/IB2024/051686

§ 371 Date: Aug. 22, 2025

Related U.S. Application Data
(60) Provisional application No. 63/486,285, filed on Feb. 22, 2023.

(51) Int. Cl. A61B 17/70; A61B 17/86

(52) U.S. Cl. CPC A61B 17/7032; A61B 17/8605; A61B 17/8685

(57) ABSTRACT

This disclosure provides for multiaxial receivers (100) for use with a tether. In an embodiment, a receiver may include first and second tether receiving passageways (6,7) that extend through first and second arm portions (3,4) of the receiver. In another embodiment, a standard multiaxial receiver (101, 102) may be used in conjunction with a tether receiving adapter (60, 70) including an upper clamp portion (63, 73) and a lower clamp portion (64, 74). The tether receiving adapter may be slipped over the standard multiaxial receiver and secured to the multiaxial receiver by engaging a nut (30) with a proximal portion of a multi-diameter set screw (20). The distal portion of the multi-diameter set screw may engage with a corresponding interior thread pattern (25) of the first and second arms to capture a longitudinal rod (40) extending therebetween. The tether receiving adapter may include at least one passageway (61, 62) for receiving a tether. In another embodiment, the tether receiving adapter may include a single passageway (61).

### FIELD

This application claims the benefit of U.S. Provisional Patent Application Ser. No. 63/486,285, filed 22 Feb. 2023, the entire content of which is incorporated herein by reference.

The present technology is generally related to implants that may be used in spinal fixation surgery. More particularly, the disclosure herein may be directed to implants and implant systems having apertures for passing of a tether, ligature, and/or artificial ligament tape to further stabilize and support the implant and patient anatomy. Disclosed implants may be multiaxial receivers that may pop on to a bone anchor, bone screw, pedicle screw, or cortical screw. Additionally, various implants may have at least one aperture for passing of a tether therethrough and an immobilization means to secure the tether to the implant.

### BACKGROUND

There are a variety of structures and methods for treating one or more degenerated, deformed or damaged vertebral stages of a patient's spinal column by means of internal spinal fixation. Typically, this involves the attachment of a spinal implant system to provide a construct that is attached to two or more adjacent vertebrae to support and stabilize the vertebrae in order to allow them to fuse together in a stationary relationship relative to each other. Spinal fusion constructions typically include bone screws and longitudinal support members or rods that are attached to the bone screws and together they may fix the position of the adjacent vertebrae to which they are attached.

The related art teaches various anchoring devices that are attached to a construct and allow the surgeon to fasten a tether and/or ligament tape to the construct. However, these specialized anchoring devices result in a construct that adds them as one or more additional devices that must be included in the construct. The additional devices increase the space occupied by the construct, require additional manipulation by the surgeon, and must be taken into account. Additionally, these constructs are typically uniaxial, have a relatively high height, consume additional space along the rod, and therefore do not provide adequate adjustability for a surgeon.

The term “tether” is used in a generic sense to refer to a type of cordage that is available for surgeons to perform various procedures for use with disclosed implant embodiments. Typically, these structures may be elongated structures that are flexible so that they bend easily (with the application of relatively little force) but also have a strong resistance to being stretched longitudinally by a substantial pulling force. For example, these structures have a relatively high tensile strength and relatively low compressive strength.

### SUMMARY

This disclosure generally relates to receivers such as multiaxial receivers for performing a surgery to a spinal column of a patient. Various multiaxial receivers may have at least one aperture for receiving a tether therein and the tether may be wrapped around various portions of a vertebrae (pedicle, lamina, spinous process, etc.) to further stabilize the patient's spinal column.

In one aspect, the present disclosure provides for a multiaxial receiver including a body having a U-shaped cavity configured to receive a longitudinal rod therein and a lower cavity configured to couple to a bone screw. In various embodiments, the body may extend in a first direction along a longitudinal axis and extend in a transverse direction along a widthwise axis. In various embodiments, the body may include a first arm portion and a second arm portion which together define the U-shaped cavity, the first arm portion may include a first passageway having a first aperture extending in the transverse direction and a first slotted opening exposing the first passageway in the first direction, and the second arm portion may include a second passageway having a second aperture extending therethrough in the transverse direction and a second slotted opening exposing the second passageway. The first and second passageways may each be configured to receive at least one tether therein. In various embodiments, a clamp for immobilizing the at least one tether may be provided and the clamp may include a first protrusion and a second protrusion opposite the first protrusion. The receiver may further include a first threaded member configured to be disposed in the U-shaped cavity such that a proximal portion of the first threaded member extends above a top surface of the first arm portion and a top surface of the second arm portion in the first direction. The receiver may further include a second threaded member configured to engage with the first threaded member such that the second threaded member is movable in the first direction to urge the clamp into a tightened position where the first protrusion extends into the first slotted opening and the second protrusion extends into the second slotted opening.

In another aspect, the disclosure provides for a multiaxial receiver and a tether adapter assembly. The multiaxial receiver may include a body having a U-shaped cavity configured to receive a longitudinal rod therein and a lower cavity configured to couple to a bone screw, and the body may extend in a first direction along a longitudinal axis and extend in a transverse direction along a widthwise axis. In various embodiments, the body may include a first arm portion and a second arm portion, which together define the U-shaped cavity. In various embodiments, the receiver may include a clamp assembly including an upper clamp portion configured to mate with a lower clamp portion. The lower clamp portion may include a first passageway having a first aperture extending in the transverse direction and a first slotted opening exposing the first passageway in the first direction, and a second passageway having a second aperture extending therethrough in the transverse direction and a second slotted opening exposing the second passageway in the first direction. For example, the first passageway may be configured to receive at least one tether therein and the second passageway may be configured to receive the at least one tether therein. In various embodiments, the upper clamp portion may include a first protrusion and a second protrusion disposed opposite the first protrusion, for example. In various embodiments, a first threaded member may be configured to be disposed in the U-shaped cavity such that a proximal portion of the first threaded member extends above a top surface of the first arm portion and a top surface of the second arm portion in the first direction. In various embodiments, a second threaded member may be configured to engage with the first threaded member such that the second threaded member is movable in the first direction thereby urging the clamp into a tightened position where the first protrusion extends into the first slotted opening and the second protrusion extends into the second slotted opening.

In another aspect, the disclosure provides for a multiaxial receiver and a tether adapter assembly. The multiaxial receiver may include a body having a U-shaped cavity configured to receive a longitudinal rod therein and a lower cavity configured to couple to a bone screw, and the body may extend in a first direction along a longitudinal axis and extend in a transverse direction along a widthwise axis. The body may include a first arm portion and a second arm portion together defining the U-shaped cavity. In various embodiments, the receiver may include a clamp assembly including an upper clamp portion configured to mate with a lower clamp portion. In various embodiments, the lower clamp portion may include a passageway having a first aperture extending in the transverse direction and a slotted opening exposing the passageway in the first direction. In various embodiments, the upper clamp portion may include a protrusion having a size and shape corresponding to a size and shape of the slotted opening, for example. In various embodiments, a first threaded member may be configured to be disposed in the U-shaped cavity such that a proximal portion of the first threaded member extends above a top surface of the first arm portion and a top surface of the second arm portion in the first direction. In various embodiments, a second threaded member may be configured to engage with the first threaded member such that the second threaded member is movable in the first direction thereby urging the clamp into a tightened position where the first protrusion extends into the first slotted opening and the second protrusion extends into the second slotted opening.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of a first multiaxial receiver embodiment.

FIG. 2 is a side view of the multiaxial receiver embodiment of FIG. 1.

FIG. 3 is a first exploded parts view of the multiaxial receiver embodiment of FIG. 1.

FIG. 4 is a second exploded parts view of the multiaxial receiver embodiment of FIG. 1.

FIG. 5 is a perspective view of a second multiaxial receiver embodiment.

FIG. 6 is a side view of the multiaxial receiver embodiment of FIG. 5.

FIG. 7 is a first exploded parts view of the multiaxial receiver embodiment of FIG. 5.

FIG. 8 is a second exploded parts view of the multiaxial receiver embodiment of FIG. 5.

FIG. 9 is an exploded parts view of select components of the multiaxial receiver embodiment of FIG. 5 from a top-down perspective.

FIG. 10 is an assembled parts perspective view of the components shown in FIG. 9.

FIG. 11 is a perspective view of a third multiaxial receiver embodiment.

FIG. 12 is a side view of the multiaxial receiver embodiment of FIG. 11.

FIG. 13 is a first exploded parts view of the multiaxial receiver embodiment of FIG. 11.

FIG. 14 is a second exploded parts view of the multiaxial receiver embodiment of FIG. 11, with the perspective from the side of the embodiment.

FIG. 15 is an exploded parts view of select components of the multiaxial receiver embodiment of FIG. 11 from a top-down perspective.

FIG. 16 is an assembled parts perspective view of the components shown in FIG. 15.

### DETAILED DESCRIPTION

Embodiments of the present disclosure relate generally, for example, to spinal stabilization systems including a receiver such as, for example, a multiaxial receiver having means for passing a tether therethrough and an immobilization means for securing the tether relative to the multiaxial receiver. Embodiments of the devices and methods are described below with reference to the Figures.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes or methods of assembly or installation. The term “substantially” may be used herein to emphasize this meaning, particularly when the described embodiment has the same or nearly the same functionality or characteristic, unless the context or other statements clearly indicate otherwise. The terms “upper” and “lower” may be used to describe the spatial relationship of one component relative to another component from an ordinary viewing angle.

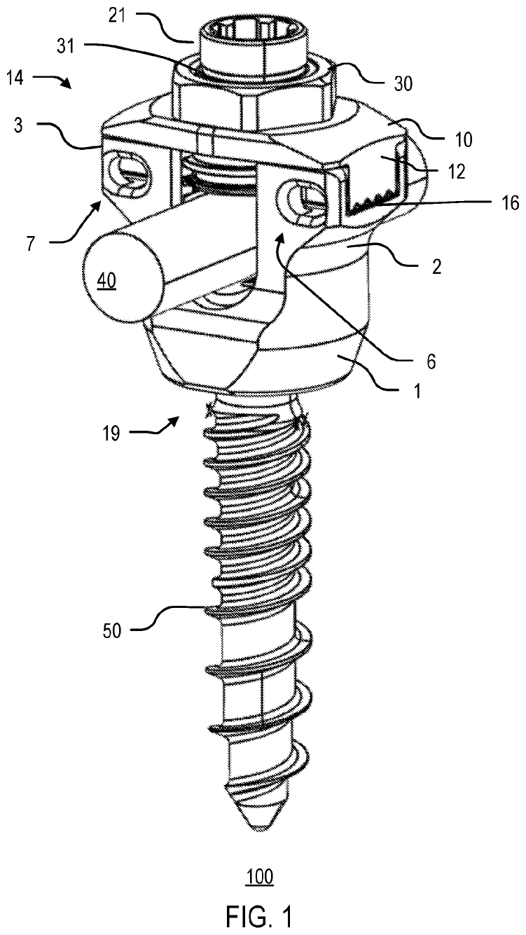

Referring to FIGS. 1-16 various multiaxial receivers 100, 101, and 102 for use with a tether are disclosed. The components of the disclosed embodiments can be fabricated from biologically acceptable materials suitable for medical applications, including metals, synthetic polymers, ceramics and bone material and/or their composites. For example, the components, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, composites of metals and calcium-based ceramics, composites of PEEK and calcium based ceramics, composites of PEEK with resorbable polymers, totally resorbable materials, such as for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplatohe, polyacetic acid or polylactide and their combinations.

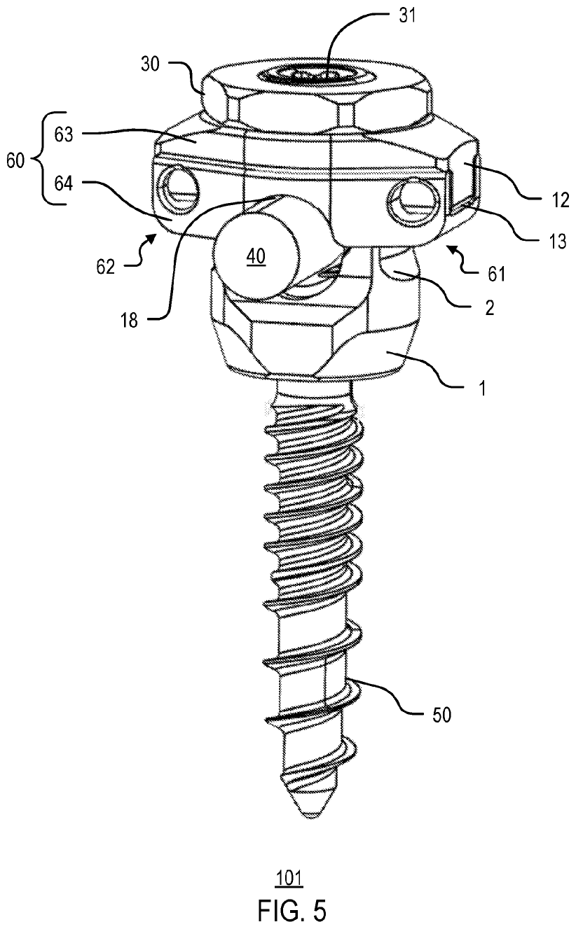

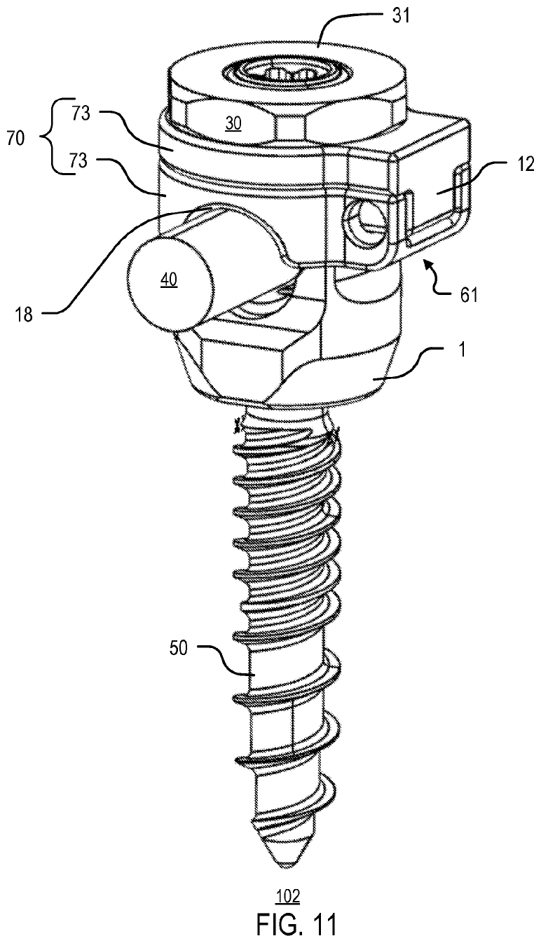

FIGS. 1-4 illustrate a first multiaxial receiver 100, FIGS. 5-10 illustrate a second multiaxial receiver 101, and FIGS. 11-16 illustrate a third multiaxial receiver 102.

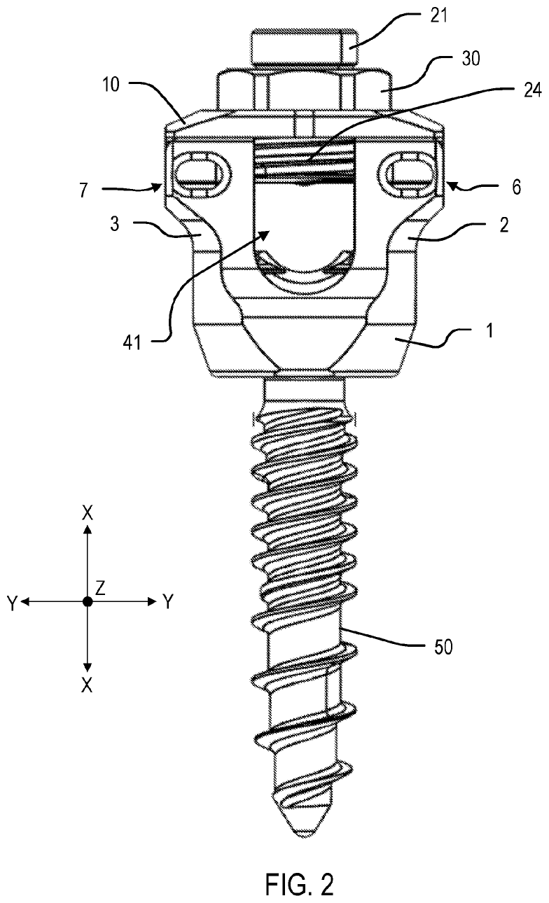

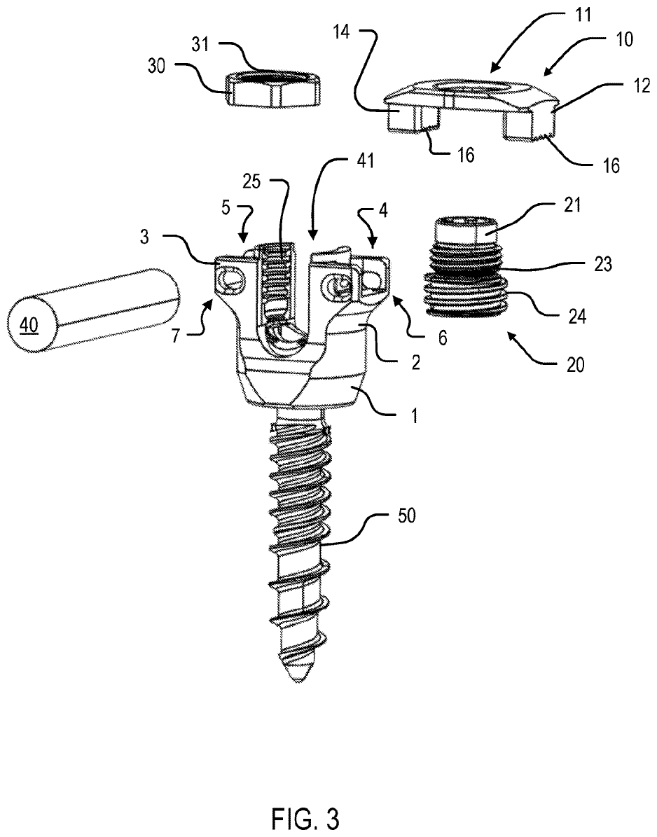

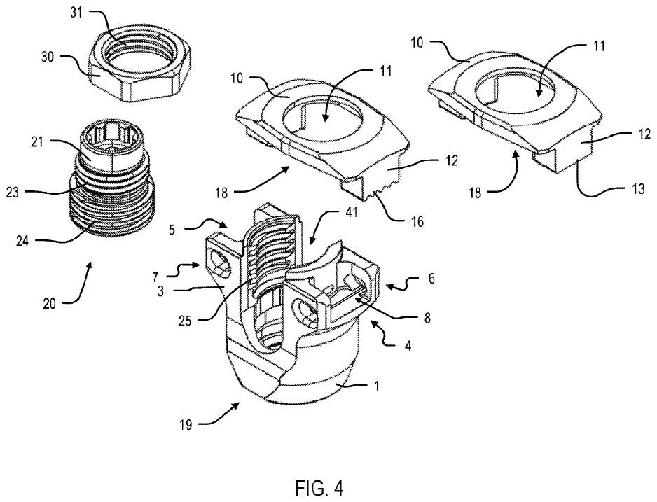

Referring generally to FIGS. 1-4 a first multiaxial receiver 100 is disclosed. FIG. 1 is a perspective view of the multiaxial receiver 100, FIG. 2 is a side view of the multiaxial receiver, FIG. 3 is an exploded view of the multiaxial receiver, and FIG. 4 is a partial parts exploded parts view. In various embodiments, receiver 100 may include a body 1 having a lower cavity 19 for coupling with the head of a bone screw 50 that may orient the receiver at a multitude of angles. Receiver 100 may include a U-shaped cavity 41 for receiving a longitudinal rod 40 therein. With reference to the side view of FIG. 2, the body 1 may extend in a vertical direction (the X axis) along a longitudinal axis and extend in a first horizontal direction (the Y axis) and a second horizontal direction (the Z axis) along a widthwise axis. In some embodiments, the vertical direction may be referred to as a first direction and the horizontal direction may be referred to a transverse direction that is substantially perpendicular to the first direction.

In the disclosed embodiment, the body 1 may include a first arm portion 2 and a second arm portion 3 that together define the U-shaped cavity 41. In the example embodiment, the U-shaped cavity 41 may include a thread form 25 on each side of the U-shaped cavity for mating with a first threaded member 20 that secures the longitudinal rod 40 from above as would be understood by a person of ordinary skill in the art. In this embodiment, a first threaded member 20 is a set screw that may be configured for disposal within the U-shaped cavity 41 of the body 1 such that the distal portion of the first threaded member 20 directly contacts the longitudinal rod 40 and a proximal portion of the first threaded member 20 extends above the top surface of the first arm portion 2 and a top surface of the second arm portion 3 in the vertical direction.

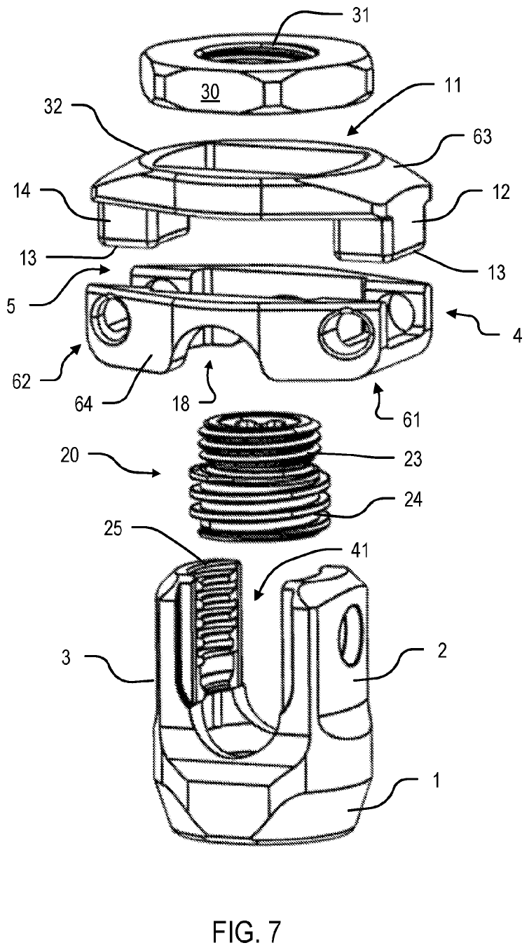

As seen best in FIG. 2, the first arm portion 2 and the second arm portion 3 are relatively wide when compared to a non-extended receiver body 1 shown in the embodiments of FIGS. 7-8 and 13-14. For example, the first arm portion 2 may include an extended and/or widened arm portion that is wide enough to accommodate a first passageway 6 having a first aperture extending in a horizontal direction. Likewise, the second arm portion 3 may include an extended and/or widened arm portion that is wide enough to accommodate a second passageway 7 having a second aperture extending in the horizontal direction. In this embodiment, the first passageway 6 and second passageway 7 extend in parallel directions offset on opposite sides of the U-shaped cavity 41. In the example embodiment, the first arm portion 2 includes a first slotted opening 4 exposing the first passageway 6 from above in the vertical direction and the second arm portion 3 includes a second slotted opening 5 exposing the second passageway 7 from above in the vertical direction. In various embodiments, the first and second passageways 6, 7 are each configured to receive at least one tether therein. For example, the first passageway 6 may receive a first tether therein and the second passageway 7 may receive a second tether therein. In some embodiments, a surgeon may utilize a single tether and make a first pass through the first passageway 6 and then a second pass through the second passageway 7 (or vice versa). Thereafter, the surgeon may immobilize the tether with respect to the body 1 by securing a clamp 10 as will be explained in further detail below.

In various embodiments, receiver 100 may include a clamp 10 having a central aperture 11, a first protrusion 12, and a second protrusion 14 disposed opposite from the first protrusion 12. With reference to FIG. 4, the first and second protrusions 12, 14 may be shaped like rectangular blocks that include a smooth bottom surface 13 or a high friction bottom surface 16 that may allow the respective type of protrusion to grip the tether passing through the first and second passageways 6, 7. As used herein, a “high friction” surface is used in its broadest sense to include any texture or feature that may increase a coefficient of friction, e.g., teeth, spikes, chamfered teeth, dimples, shot peening, etc. to facilitate the immobilization of a tether. For example, as shown in FIG. 1, the clamp 10 may be positioned above the body 1 such that the first protrusion 12 extends into the first passageway 6 and the second protrusion 14 extends into the second passageway 7 from above. For example, a size and shape of the first and second protrusions may correspond to a size and shape of the first and second slots 4, 5 (which communicate with the first and second passageways 6, 7 from above).

With reference to FIG. 2 and FIG. 3, the central aperture 11 of the clamp 10 may be configured to permit the proximal portion of the first threaded member 20 to extend therethrough such that the proximal portion of the first threaded member 20 is exposed and can secure to the second threaded member 30. In various embodiments, the second threaded member 30 is a nut that may be configured to engage with the proximal portion of the first threaded member 20 such that when the second threaded member 30 is rotated in a first direction the second threaded member 30 is movable in the vertical direction along a corresponding length of the first threaded member 20.

In an alternate embodiment, the first threaded member 20 may be a bolt having an outside thread pattern that is configured to threadably engage with the interior thread pattern 25 of the first and second arm portions 2, 3 (not illustrated). For example, the bolt may have a hollow central aperture with a corresponding thread pattern that is configured to receive a set screw. The set screw may be rotated such that it travels in a linear direction towards the longitudinal rod 40. In this way, a bolt having a corresponding set screw for threading to an interior portion of the bolt may also be utilized to position the clamp 10 into a tightened position.

In this embodiment, first threaded member 20 is a multi-diameter breakoff set screw including a break-off portion 21, a first diameter thread pattern 23 and a second diameter thread pattern 24. For example, the first diameter thread pattern 23 corresponds to a smaller outside diameter of the set screw and the second diameter thread pattern 24 corresponds to a larger outside diameter of the set screw. In an installed position where the first threaded member 20 directly contacts the longitudinal rod 40, the proximal portion of the first threaded member 20 (including the break-off portion 21 and the first diameter thread pattern 23) may extend above an upper surface of the clamp 10. In this way, an interior thread pattern 31 of the second threaded member 30 can engage with the first diameter thread pattern 23 of the first threaded member 20. Accordingly, when the second threaded member 30 is rotated it will advance downward in the vertical direction thereby urging the clamp 10 into a secured position in which the first and second protrusions 12, 14 will immobilize a tether passing through either one or both of the first and second passageways 6, 7. Thereafter, the break-off portion 21 may be sheared off with any suitable tool to reduce a height of the receiver 100.

As seen best in FIG. 4, a valley 8 may be included on a bottom bearing surface of the first passageway 6 and/or second passageway 7. In various embodiments, the valley 8 may form a gap space between a bottom surface of the first and second protrusions 12, 14 and the bearing surface of the first and second passageways 6, 7. The valley 8 may have the advantage of providing a pressure relief for a tether such that when the clamp 10 is secured in the fully tightened position the tether will not be damaged or cut due to excessive tightening. In the example embodiment, the longitudinal rod 40 may be oriented in the horizontal direction in a direction that is substantially parallel with an extension direction of the first passageway 6 and the second passageway 7. However, in other embodiments, the body 1 may be contoured in such a way as to orient the longitudinal rod 40 off angle and/or at an angle with respect to the extension directions of the first passageway 6 and/or the second passageway 7 (not illustrated).

In a method of installation, a first step of installation may include initially securing the body 1 to the bone screw 50 and thereafter securing the longitudinal rod 40 within the U-shaped cavity 41 by tightening the first threaded member 20. Once the body 1 and the longitudinal rod 40 are secured in position, a surgeon may pass a tether through either one or both of the passageways 6, 7 of the body 1. For example, a surgeon may pass a tether through first passageway 6 and wrap the tether around a spinous process portion of a vertebra and then pass the tether back through second passageway 7. Thereafter, the surgeon may immobilize the tether with respect to the body 1 by rotating the second threaded member 30 along the first diameter thread pattern 23 of first threaded member 20 such that the first and second protrusions 12, 14 will bear down upon the tether from above by passing through the first slotted opening 4 and second slotted opening 5. It shall be appreciated that this example is non-limiting, and that other methodologies of installation and installation paths of the tether are contemplated. For example, it shall be understood that in some embodiments, the surgeon may pass the tether through an aperture of the body 1 before securing the body 1 to the bone screw 50 and/or longitudinal rod 40. In summary, embodiments in accordance with the principles herein provide a surgeon with the capability of performing different installation sequences, e.g., the tether may be passed either before, after, or even concurrently with securing the body 1 to the bone screw 50.

Referring generally to FIGS. 5-10, a second multiaxial receiver 101 for receiving a tether is disclosed. Second multiaxial receiver 101 may include the same, similar, and/or substantially the same features and functionality as explained above with respect to receiver 101. Accordingly, like numbering of parts will be maintained where feasible and duplicative description will be omitted or only briefly described.

This embodiment may differ in that the arm portions 2, 3 do not have extended portions for accommodating a tether receiving passageway. Rather, in this embodiment a tether receiving adapter assembly is utilized with a standard width multiaxial receiver. As used herein, a standard width multiaxial receiver is used to describe a receiver in which the outside width of the receiver measured through the first arm portion 2 and second arm portion 3 remains substantially constant. Stated another way, a first distance measured in the horizontal direction from a central vertical axis bisecting the receiver into two equal halves to an outside side surface of the first arm portion is equal to a second distance measured in the horizontal direction from the central vertical axis to a side surface of the second arm portion.

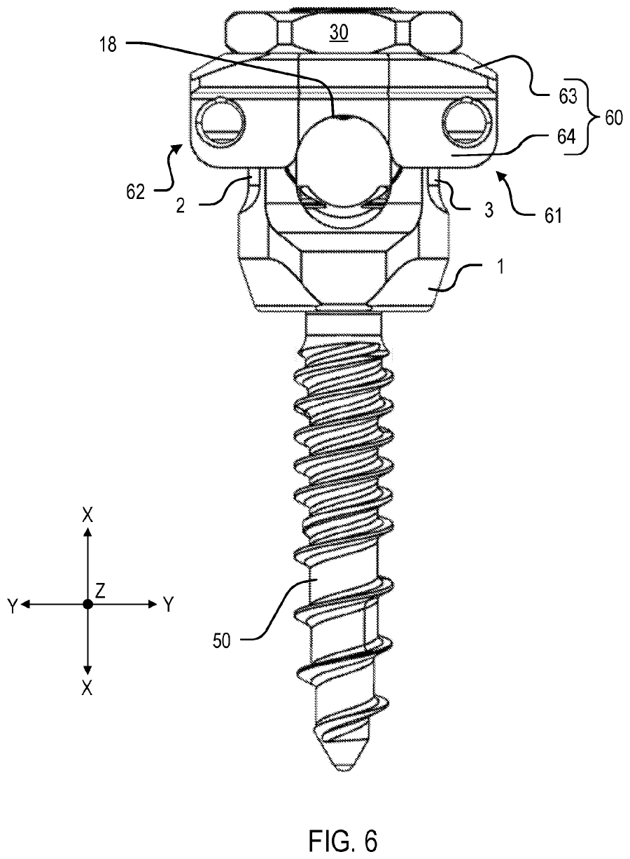

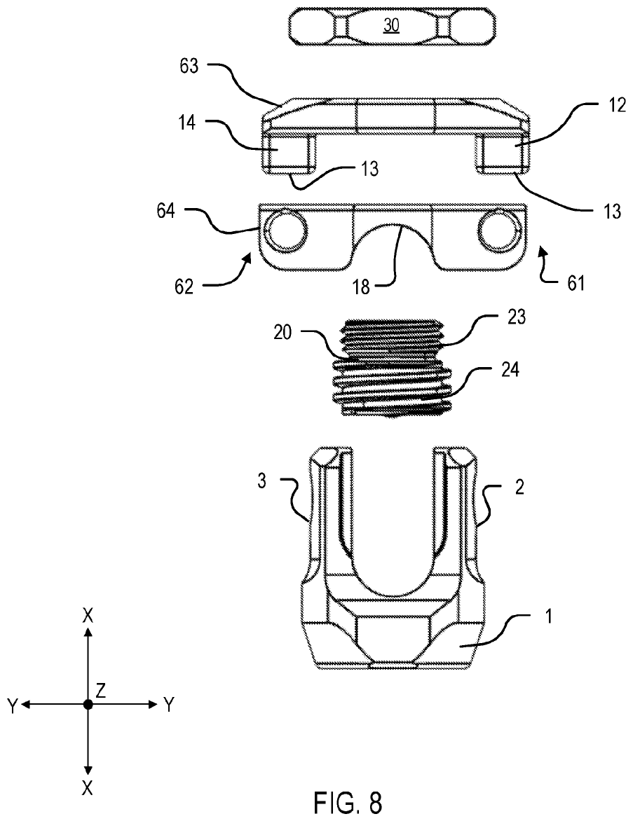

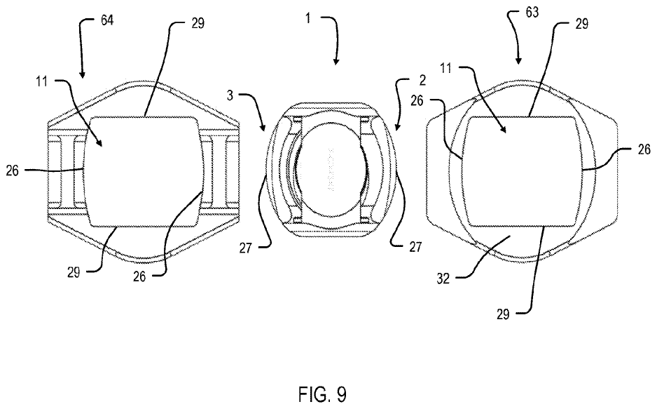

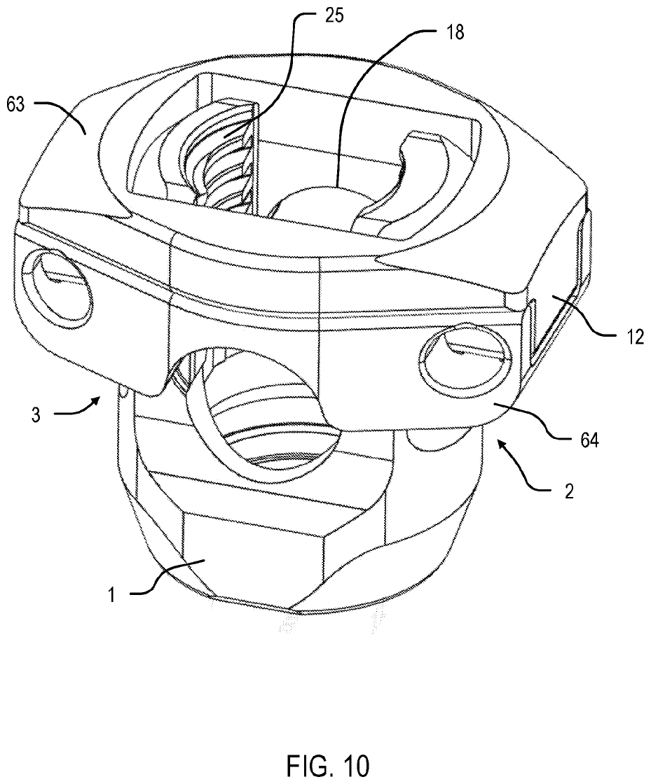

FIG. 5 is a perspective view of the second multiaxial receiver embodiment 101 and FIG. 6 is a side view of the second multiaxial receiver embodiment 101. FIGS. 7 and 8 are exploded parts views of the second multiaxial receiver embodiment 101. FIG. 9 is an exploded parts view of a tether adapter assembly, which in this embodiment is a two-part clamp assembly 60. FIG. 10 is an assembled parts perspective view of the components shown in FIG. 9.

Receiver 101 may include a body 1 having a U-shaped cavity 41 configured to receive a longitudinal rod 40 therein. The body 1 may include a first arm portion 2 and a second arm portion 3, which together form the U-shaped body 1. The U-shaped cavity 41 may include a thread form 25 to receive a first threaded member 20 therein to secure the longitudinal rod 40 there below.

As seen best in FIG. 7, first threaded member 20 may be a multiple diameter set screw that includes include first thread pattern 23 and a second diameter thread pattern 24. In this example, the minor diameter portion corresponding to the first diameter thread pattern 23 may extend above the upper most surfaces of the first arm portion 2 and the second arm portion 4. In this way, the first thread pattern 23 may engage with the interior thread pattern 31 of the second threaded member 30 similarly as explained above. The major diameter portion corresponding to the second diameter thread pattern 24 may thread to the thread form 25 on the interior of the first arm portion 2 and second arm portion 3. In this way, a bottom most surface of the first threaded member 20 corresponding to the major diameter portion may directly contact the longitudinal rod 40.

In this embodiment, receiver 101 has a tether adapter assembly including a two-part clamp assembly 60 having an upper clamp portion 63 and a lower clamp portion 64. The upper clamp portion 63 may be configured to mate and/or engage with a lower clamp portion 64 as will be explained in further detail below. In the example embodiment, lower clamp portion 64 may include a first passageway 61 and a second passageway 62 disposed on opposite sides of a central aperture 11. The lower clamp portion 64 may further include slotted openings 4, 5 on either side of the central aperture 11, that expose the first and second passageways 61, 62, respectively, from above. The upper clamp portion 63 may include a first protrusion 12 and second protrusion 14 on either side of the central aperture 11. These protrusions 12, 14 may have a size and shape generally corresponding to a size and shape of the slotted openings 4, 5 similarly as explained above. As seen in FIG. 7, upper clamp portion 63 may include a recessed groove 32 that is slightly larger than the central aperture 11 and is configured to allow the second threaded member 30 to rest therein. In this way, when the second threaded member 30 is rotated around the first thread pattern 23 of the first threaded member 20, a bottom surface of the second threaded member 30 slides along the recessed groove 32. This may have the advantage of helping center the second threaded member 30 and first threaded member 20 as well as decreasing a vertical height of the receiver 101.

With reference to FIGS. 9 and 10, receiver 101 has a two-part clamp assembly 60 in which the central aperture 11 is of a certain size and shape that corresponds to an ovoid rectangle such that the two-part clamp assembly 60 can be slipped over the body 1 of the receiver 101 (see FIG. 10). For example, from a top-down perspective, a size and shape of central aperture 11 corresponds to a size and shape of the body 1 measured between the first arm portion 2 and second arm portion 3. As shown in FIG. 9, the central aperture 11 of lower clamp portion 64 and upper clamp portion 63 each resemble the shape of a rectangle with curved side surfaces. For example, the central aperture 11 of the lower clamp portion 64 and upper clamp portion 63 each include a respective pair of curved surfaces 26 and a respective pair of substantially straight surfaces 29. The curved surfaces 26 have a geometry that corresponds in length and curvature to the outermost surfaces 27 of the first and second arm portions 2, 3. Similarly, the substantially straight surfaces 29 correspond in length to a distance between the first arm portion 2 and second arm portion 3. This configuration has the advantage of allowing a surgeon to use a standard width multiaxial receiver body 1. This configuration may also prevent the two-part clamp assembly 60 from rotating during tightening of the second threaded member 30. For example, the two-part clamp assembly 60 may fit snugly around the body 1 such that twisting or turning of the two-part clamp assembly 60 relative to the body 1 is prevented or suppressed.

With reference to FIG. 7 and FIG. 8, lower clamp portion 64 may include a relief contour 18 on a bottom surface thereof that is shaped like a half circle. In this example, the relief contour 18 has a size and shape that generally corresponds to the curvature of an exposed surface of the longitudinal rod 40 (e.g., a corresponding arc segment along the longitudinal rod 40). In this way, a surgeon may slip the lower clamp portion 64 over the body 1 and allow it to rest on top of the longitudinal rod 40. Thereafter, a surgeon may slip the upper clamp portion 63 over the body 1 before passing a tether through either one or both of the first and second passageways 61, 62. Once the two-part clamp assembly 60 is slipped over the body 1, the proximal portion of the first threaded member 20 may extend above the top surface of the first and second arm portions 2, 3 and the top surface of the two-part clamp assembly 60. Similarly as explained above with respect to receiver 100, the receiver 101 may include a second threaded member 30 having an interior thread pattern 31 that is configured to engage with the proximal portion of the first threaded member 20 that extends above the top surface of the two-part clamp assembly 60. For example, the interior thread pattern 31 may have a thread pitch that corresponds to the thread pitch of the first diameter thread pattern 23. In this way, once the second diameter thread pattern 24 of first threaded member 20 is engaged with the body 1 and the longitudinal rod 40 there below is appropriately captured a surgeon can begin to tighten the second threaded member 30 to lock the two-part clamp assembly 60 in place relative to the body 1. For example, when the second threaded member 30 is rotated in a first direction the second threaded member 30 moves downward in the vertical direction thereby urging the upper clamp portion 63 downward towards the lower clamp portion 64 into a locked configuration in which the protrusions 12 and 14 may extend into the passageways 61, 62 from above through the slotted openings 4, 5 to immobilize a tether extending through either one or both of the first passageway 61 and/or second passageway 62. Conversely, when the two-part clamp assembly 60 is not yet tightened via the second threaded member 30, the tether is allowed to freely pass through either of the passageways 61, 62.

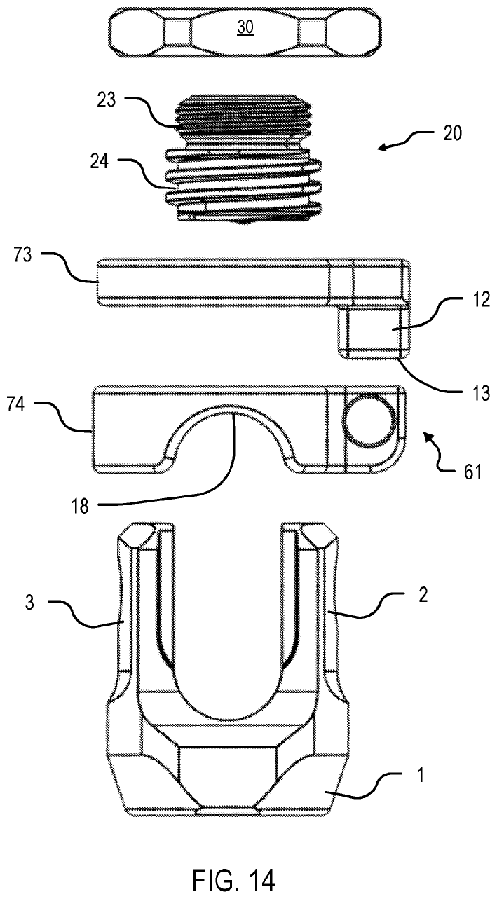

Referring generally to FIGS. 11-16 a third multiaxial receiver 102 for receiving a tether is disclosed. Third multiaxial receiver 102 may include the same, similar, and/or substantially the same features and functionality as explained above with respect to receivers 100 and 101. Accordingly, like numbering of parts will be maintained where feasible and duplicative description will be omitted or only briefly described. Multiaxial receiver 102 may be similar to receiver 101 because in this embodiment a tether receiving adapter assembly is utilized with a standard width multiaxial receiver. Multiaxial receiver 102 may be different from receiver 101 in that it only includes a single tether receiving passageway 61 (see FIGS. 11 and 14).

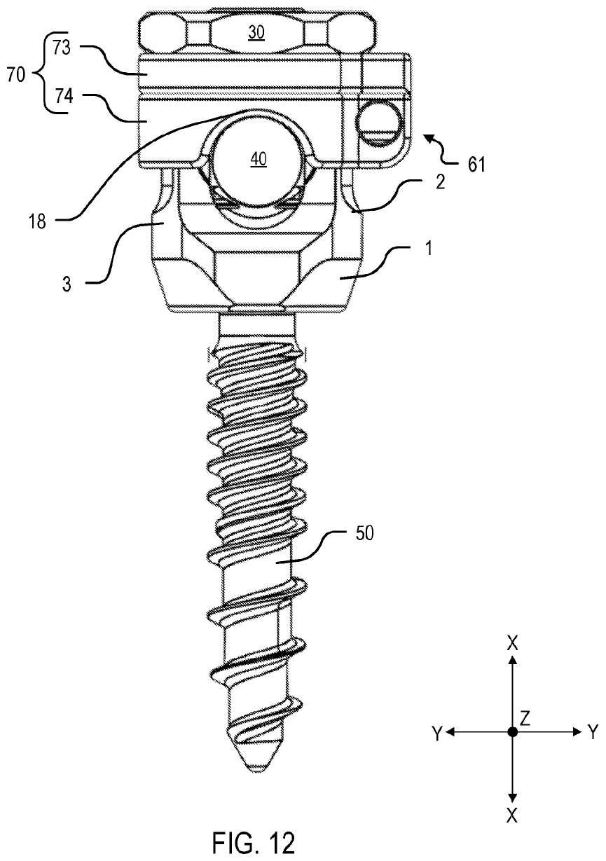

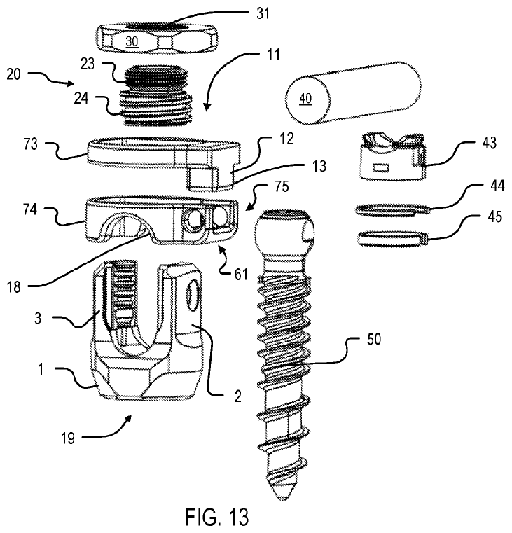

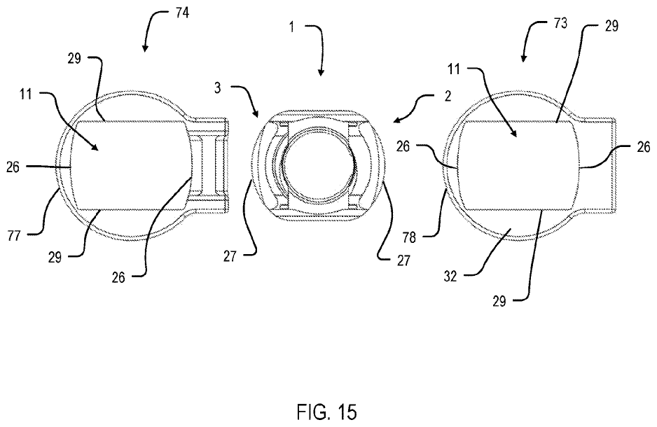

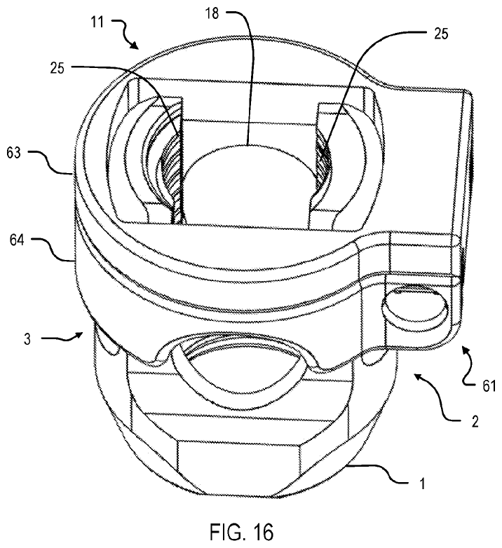

FIG. 11 is a perspective view and FIG. 12 is a side view of the multiaxial receiver 102. FIGS. 13 and 14 are exploded parts views of the multiaxial receiver 102. FIG. 15 is a top-down exploded parts view of a tether adapter assembly, which in this embodiment is a reduced width curved two-part clamp assembly 70. FIG. 16 is an assembled parts perspective view of the components shown in FIG. 15.

Referring generally to FIGS. 11-16, the multiaxial receiver 102 may include a body 1 with a U-shaped cavity 41 configured to receive a longitudinal rod 40. Similarly to the first and second receivers 100, 101, the body 1 may include a lower cavity 19 configured to couple to the head of a bone screw 50. For example, as seen best in the exploded parts view of FIG. 13, it is shown that receiver 102 (and any of the other receivers disclosed herein) may include a saddle and/or crown 43 for supporting a longitudinal rod 40 and any number of washers, annular rings 44, c-rings 45, etc. for facilitating the coupling of multiaxial receiver 102 to a bone screw 50 (see FIG. 12). For example, each of the annular rings 44, c-rings 45, etc. may be disposed in a corresponding annular groove (not shown) of the lower receiving cavity 19. In this way, multiaxial receiver 102 may be popped on to the head of a bone screw 50 simply by pressing down on receiver 102 as would be understood by a person of ordinary skill in the art.

Referring generally to FIGS. 13-14, receiver 102 may include a first threaded member 20 configured for disposal in the U-shaped cavity 41 such that the distal portion of first threaded member 20 may directly contact the longitudinal rod 40 and the proximal portion of first threaded member 20 may extend above a top surface of the first arm portion 2 and a top surface of the second arm portion 3 in the vertical direction. Receiver 102 may include a reduced width curved two-part clamp assembly 70 having an upper clamp portion 73 configured to mate with a lower clamp portion 74. The upper clamp portion 73 and lower clamp portion 74 may each slip around the first and second arm portions 2, 3 of body 1 similarly as explained above.

Similar to embodiment 101, the lower clamp portion 74 may include a relief contour 18 on a bottom surface thereof that is shaped like a half circle. In various embodiments, the lower clamp portion 74 may include a single passageway 61 having a first aperture extending in the horizontal direction for receiving a tether therein. The lower clamp portion 74 may further include a slotted opening 75 exposing the passageway 61 in the vertical direction. In this embodiment, the lower clamp portion 74 includes a curved side surface 77 on an opposite side surface from the passageway 61. Additionally, the upper clamp portion 36 includes a corresponding curved side surface 78. In various embodiments, the upper clamp portion 73 may include a protrusion 12 disposed opposite of the curved side surface 78. The protrusion 12 may have a size and shape that corresponds to a size and shape of the slotted opening 75 (see FIG. 13).

Similar to receiver 101, receiver 102 includes a second threaded member 30 with an interior thread pattern 31 that is configured to engage with the proximal portion of the first threaded member 20 that extends above the top surface of the curved two-part clamp assembly 70. For example, the interior thread pattern 31 may have a thread pitch that corresponds to the thread pitch of the first thread pattern 23. In this way, once the second thread pattern 24 of first threaded member 20 is engaged with the body 1 and the longitudinal rod 40 there below is in a tightened position a surgeon can begin to tighten the second threaded member 30 to lock the curved two-part clamp assembly 70 in place relative to the body 1. For example, when the second threaded member 30 is rotated in a first direction the second threaded member 30 moves downward in the vertical direction thereby urging the upper clamp portion 73 downward towards the lower clamp portion 74 into a locked configuration in which the protrusion 12 may extend into the passageway 61 from above to immobilize a tether extending through passageway 61. Conversely, when the two-part clamp 70 is not yet tightened via the second threaded member 30, the tether is allowed to freely pass through the passageway 61.

As depicted in FIGS. 15-16, receiver 102 has a curved two-part clamp assembly 70 in which the central aperture 11 is of a certain size and shape that corresponds to an ovoid rectangle such that the two-part clamp assembly 70 can be slipped over the body of the receiver 1 (see FIG. 16). For example, from a top-down perspective, a size and shape of central aperture 11 corresponds to a size and shape of the body 1 measured between the first arm portion 2 and second arm portion 3. As shown in FIG. 15, the central aperture 11 of lower clamp portion 74 and upper clamp portion 73 each resemble the shape of a rectangle with curved side surfaces. For example, the central aperture 11 of the lower clamp portion 74 and upper clamp portion 73 each include a respective pair of curved surfaces 26 and a respective pair of substantially straight surfaces 29. The curved surfaces 26 have a geometry that corresponds in length and curvature to the outermost surfaces 27 of the first and second arm portions 2, 3. Similarly, the substantially straight surfaces 29 correspond in length to a distance between the first arm portion 2 and second arm portion 3. This configuration has the advantage of allowing a surgeon to use a standard width multiaxial receiver body 1. This configuration may also prevent the curved two-part clamp assembly 70 from rotating during tightening of the second threaded member 30. For example, the two-part clamp 70 may fit snugly around the body 1 such that twisting or turning of the two-part clamp 70 relative to the body 1, first arm portion 2, second arm portion 3, is prevented and/or suppressed.

This embodiment may have additional advantages in select surgical applications due to the decreased width of the curved two-part clamp assembly 70 relative to the two-part clamp assembly 60 of receiver 101. For example, the curved side surfaces 77, 78 more closely approximate the curved side surfaces 27 of first arm portion 2. In this way, a width of the curved two-part clamp assembly 70 may be reduced to have additional clearance.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

1-15. (canceled)

16. A multiaxial receiver, comprising:
a body having a U-shaped cavity configured to receive a longitudinal rod therein and a lower cavity configured to couple to a bone screw, the body extending in a first direction along a longitudinal axis and extending in a transverse direction along a widthwise axis;
a first arm portion and a second arm portion together defining the U-shaped cavity, the first arm portion including a first passageway having a first aperture extending in the transverse direction and a first slotted opening exposing the first passageway in the first direction, the second arm portion including a second passageway having a second aperture extending therethrough in the transverse direction and a second slotted opening exposing the second passageway in the first direction, the first passageway and the second passageway each being configured to receive at least one tether therein;
a clamp including a first protrusion and a second protrusion opposite the first protrusion;
a first threaded member configured to be disposed in the U-shaped cavity such that a proximal portion of the first threaded member extends above a top surface of the first arm portion and a top surface of the second arm portion in the first direction; and
a second threaded member configured to engage with the first threaded member such that the second threaded member is movable in the first direction to urge the clamp into a tightened position where the first protrusion extends into the first slotted opening and the second protrusion extends into the second slotted opening.

17. The multiaxial receiver of claim 16, wherein:
the first threaded member is a set screw configured to be disposed in the U-shaped cavity such that a distal portion of the set screw directly contacts the longitudinal rod and a proximal portion of the set screw extends through a central cavity of the clamp and above a top surface of the first arm portion and a top surface of the second arm portion in the first direction, and
the second threaded member is a nut configured to engage with the proximal portion of the set screw.

18. The multiaxial receiver of claim 17, wherein the set screw is a multi-diameter set screw having a first major diameter defining a first thread pattern that corresponds to a thread pattern of the U-shaped cavity and a second major diameter defining a second thread pattern corresponding to an interior thread pattern of the nut.

19. The multiaxial receiver of claim 18, wherein the set screw comprises a breakoff portion.

20. The multiaxial receiver of claim 16, wherein:
the first aperture comprises a valley extending in the transverse direction such that in the tightened position a gap space exists between a bottom surface of the first passageway and a bottom surface of the first protrusion, and
the gap space is configured to reduce the likelihood of cutting of at least one tether in the tightened position.

21. The multiaxial receiver of claim 16, wherein the first protrusion is shaped like a rectangular block and the second protrusion is shaped like a rectangular block.

22. The multiaxial receiver of claim 21, wherein a bottom surface of the first protrusion and a bottom surface of the second protrusion each comprise a high friction surface configured to grip the at least one tether.

23. The multiaxial receiver of claim 16, wherein:
the first arm portion includes a first extended portion and the second arm portion includes a second extended portion opposite the first extended portion, the first extended portion protruding away from the U-shaped cavity in the transverse direction and the second extended portion extending away from the U-shaped cavity in the transverse direction, and
the first passageway extends through the first extended portion and the second passageway extends through the second extended portion.

24. A multiaxial receiver and a tether adapter assembly, comprising:
a body having a U-shaped cavity configured to receive a longitudinal rod therein and a lower cavity configured to couple to a bone screw, the body extending in a first direction along a longitudinal axis and extending in a transverse direction along a widthwise axis, the body including a first arm portion and a second arm portion together defining the U-shaped cavity;
a clamp assembly including an upper clamp portion configured to mate with a lower clamp portion
the lower clamp portion including a first passageway having a first aperture extending in the transverse direction and a first slotted opening exposing the first passageway in the first direction, and a second passageway having a second aperture extending therethrough in the transverse direction and a second slotted opening exposing the second passageway in the first direction, the first passageway being configured to receive at least one tether therein and the second passageway being configured to receive the at least one tether therein;
the upper clamp portion including a first protrusion and a second protrusion disposed opposite the first protrusion;
a first threaded member configured to be disposed in the U-shaped cavity such that a proximal portion of the first threaded member extends above a top surface of the first arm portion and a top surface of the second arm portion in the first direction; and
a second threaded member configured to engage with the first threaded member such that the second threaded member is movable in the first direction thereby urging the clamp into a tightened position where the first protrusion extends into the first slotted opening and the second protrusion extends into the second slotted opening.

25. The multiaxial receiver of claim 24, wherein:
the first threaded member is a set screw configured to be disposed in the U-shaped cavity such that a distal portion of the set screw directly contacts the longitudinal rod and a proximal portion of the set screw extends above a top surface of the first arm portion and a top surface of the second arm portion in the first direction, and
the second threaded member is a nut configured to engage with the proximal portion of the set screw extending above the top surface of the clamp assembly such that when the nut is rotated in a first direction the nut is movable in the first direction thereby urging the clamp assembly into a tightened position.

26. The multiaxial receiver of claim 24, wherein, in a cross-section view, the first aperture comprises a circular shape such that in the tightened position a gap space exists between a bottom surface of the first passageway and a bottom surface of the first protrusion, and
wherein the gap space is configured to reduce the likelihood of cutting of the at least one tether in the tightened position.

27. The multiaxial receiver of claim 24, wherein a bottom surface of the first protrusion and a bottom surface of the second protrusion each comprise a high friction surface configured to grip the at least one tether.

28. The multiaxial receiver of claim 24, wherein:
the upper clamp portion comprises a first central aperture and the lower clamp portion comprises a second central aperture, and
the second central aperture is configured to surround an upper region of the first arm portion and second arm portion.

29. The multiaxial receiver of claim 24, wherein the longitudinal axis extends in the first direction through a centerline of the body that bisects the body into substantially symmetrical halves, and
wherein a first distance measured in the transverse direction from the longitudinal axis to an exposed side surface of the first arm portion is equal to a second distance measured in the transverse direction from the longitudinal axis to an exposed side surface of the second arm portion.

30. A multiaxial receiver and a tether adapter assembly, comprising:
a body having a U-shaped cavity configured to receive a longitudinal rod therein and a lower cavity configured to couple to a bone screw, the body extending in a first direction along a longitudinal axis and extending in a transverse direction along a widthwise axis, the body including a first arm portion and a second arm portion together defining the U-shaped cavity;
a clamp assembly including an upper clamp portion configured to mate with a lower clamp portion;
the lower clamp portion including a passageway having a first aperture extending in the transverse direction and a slotted opening exposing the passageway in the first direction;
the upper clamp portion including a protrusion having a size and shape corresponding to a size and shape of the slotted opening; and
a first threaded member configured to be disposed in the U-shaped cavity such that a proximal portion of the first threaded member extends above a top surface of the first arm portion and a top surface of the second arm portion in the first direction;
a second threaded member configured to engage with the first threaded member such that the second threaded member is movable in the first direction thereby urging the clamp into a tightened position where the first protrusion extends into the first slotted opening and the second protrusion extends into the second slotted opening.

31. The multiaxial receiver of claim 30, wherein:
the first threaded member is a set screw configured to be disposed in the U-shaped cavity such that a distal portion of the set screw directly contacts the longitudinal rod and a proximal portion of the set screw extends above a top surface of the first arm portion and a top surface of the second arm portion in the first direction, and
the second threaded member is a nut configured to engage with the proximal portion of the set screw extending above the top surface of the clamp assembly such that when the nut is rotated in a first direction the nut is movable in the first direction thereby urging the clamp assembly into a tightened position.

32. The multiaxial receiver of claim 30, wherein, in a cross-section view, the first aperture comprises a circular shape such that in the tightened position a gap space exists between a bottom surface of the passageway and a bottom surface of the protrusion, and
wherein the gap space is configured to reduce the likelihood of cutting of the tether in the tightened position.

33. The multiaxial receiver of claim 30, wherein a bottom surface of the protrusion comprises a high friction surface configured to grip the tether.

34. The multiaxial receiver of claim 30, wherein:
the upper clamp portion comprises a first central aperture and the lower clamp portion comprises a second central aperture, and
the second central aperture is configured to surround an upper region of the first arm portion and second arm portion.

35. The multiaxial receiver of claim 30, wherein the longitudinal axis extends in the first direction through a centerline of the body that bisects the body into substantially symmetrical halves, and
wherein a first distance measured in the transverse direction from the longitudinal axis to an exposed side surface of the first arm portion is equal to a second distance measured in the transverse direction from the longitudinal axis to an exposed side surface of the second arm portion.
